(19) United States
(12) Patent Application Publication
(10) Pub. No.: US 2024/0091466 A1
(43) Pub. Date: Mar. 21, 2024

(54) COOLDOWN ALERT SYSTEM

(71) Applicant: Altria Client Services LLC, Richmond, VA (US)

(72) Inventors: Zack W. BLACKMON, Richmond, VA (US); Thomas TRAN, Richmond, VA (US); Terry BACHE, Richmond, VA (US); Jarrett KEEN, Richmond, VA (US); Niall GALLAGHER, Richmond, VA (US); Yannick HOURMAND, Haslingfield (GB); Guillaume MICHEL, Cambridge (GB); Rangaraj S. SUNDAR, Midlothian, VA (US); Raymond W. LAU, Glen Allen, VA (US); Eric HAWES, Midlothian, VA (US)

(73) Assignee: Altria Client Services LLC, Richmond, VA (US)

(21) Appl. No.: 17/947,383

(22) Filed: Sep. 19, 2022

(51) Int. Cl. A61M 11/04

(52) U.S. Cl. CPC A61M 11/042; A61M 2205/3606; A61M 2205/502; A61M 2205/8206

(57) ABSTRACT

A cooldown system for a device includes at least one processor and a memory coupled to the at least one processor. The memory is configured to store instructions. The at least one processor is configured to execute the instructions to cause the cooldown system to detect when a session has ended, activate a cooldown timer to measure a cooldown time, provide an indication of a remaining time of the cooldown timer, and in response to the cooldown timer elapsing, return the device to normal operation.

### FIELD

The present disclosure relates to heat-not-burn (HNB) aerosol generating devices and capsules configured to generate an aerosol without involving a substantial pyrolysis of an aerosol-forming substrate.

### DESCRIPTION OF RELATED ART

Some electronic devices are configured to heat a plant material to a temperature that is sufficient to release constituents of the plant material while keeping the temperature below a combustion point of the plant material so as to avoid any substantial pyrolysis of the plant material. Such devices may be referred to as aerosol-generating devices (e.g., heat-not-burn aerosol-generating devices), and the plant material heated may be tobacco and/or cannabis. In some instances, the plant material may be introduced directly into a heating chamber of an aerosol generating device. In other instances, the plant material may be pre-packaged in individual containers to facilitate insertion and removal from an aerosol-generating device.

### BRIEF SUMMARY

New and useful systems, apparatuses, and methods for cooldown alert systems for aerosol-generating devices are set forth in the appended claims. Illustrative embodiments are also provided to enable a person skilled in the art to make and use the claimed subject matter.

For example, in some example embodiments, a cooldown system for a device is described. The cooldown system can include at least one processor and a memory coupled to the at least one processor. The memory can be configured to store instructions. The at least one processor can be configured to execute the instructions to cause the cooldown system to detect when a session has ended, activate a cooldown timer to measure a cooldown time, provide an indication of a remaining time of the cooldown timer, and in response to the cooldown time elapsing, return the device to normal operation.

In some example embodiments, the at least one processor can be configured to execute the instructions to cause the cooldown system to obtain a signal from a control button. The signal can indicate the device has been switched off and the session has ended.

In some example embodiments, the at least one processor can be configured to execute the instructions to cause the cooldown system to obtain a signal from a mechanism detection switch. The signal can indicate that a lid mechanism has been opened and that the session has ended.

In some example embodiments, the at least one processor can be configured to execute the instructions to cause the cooldown system to obtain a signal from a charging connection. The signal can indicate that the device is connected to a charger and that the session has ended.

In some example embodiments, the at least one processor can be configured to execute the instructions to cause the cooldown system to obtain a signal from a battery monitoring system. The signal can indicate that the device has entered a low battery state and that the session has ended.

In some example embodiments, the at least one processor can be configured to execute the instructions to cause the cooldown system to detect when the device is in a fault state. The fault state can indicate that the session has ended.

In some example embodiments, the at least one processor can be configured to execute the instructions to cause the cooldown system to display an alert icon on the user interface if a lid mechanism of the device is opened while the cooldown timer is active.

In some example embodiments, the at least one processor can be configured to execute the instructions to cause the cooldown system to display a cooldown alert icon if a lid mechanism of the device is opened while the session is underway and prior to the cooldown timer being activated.

In some example embodiments, the at least one processor can be configured to execute the instructions to cause the cooldown system to activate a refresh timer after the cooldown timer is activated. In some example embodiments, the refresh timer can be configured to measure a refresh time. The refresh timer can additionally be configured to reset if the cooldown timer is active when the refresh timer elapses. The refresh time can be less than the cooldown time. In some example embodiments, the at least one processor can be configured to execute the instructions to cause the cooldown system to refresh the timer icon when the refresh time elapses.

In some example embodiments, the at least one processor can be configured to execute the instructions to cause the cooldown system to display, on the user interface, a capsule eject icon when the cooldown time has elapsed. The capsule eject icon can indicate that a capsule of the device can be removed.

In some example embodiments, the at least one processor can be configured to execute the instructions to cause the cooldown system to activate a holdoff timer for a holdoff time and display, on the user interface, a system icon while activating the cooldown timer. In some example embodiments, the at least one processor can be configured to execute the instructions to cause the cooldown system to display the cooldown aware icon after the holdoff time elapses.

In some example embodiments, the at least one processor can be configured to execute the instructions to cause the cooldown system to activate a holdoff timer for a holdoff time and display a power off indication if the device is turned off while the cooldown timer is active. In some example embodiments, the at least one processor can be configured to execute the instructions to cause the cooldown system to display a cooldown alert icon after the holdoff timer elapses and until the cooldown timer has elapsed.

In some example embodiments, the cooldown time can be based on a length of a session of the device.

In some example embodiments, the cooldown system can further include a temperature sensor. In some example embodiments, the cooldown time can be based on a temperature measured by the temperature sensor.

In some example embodiments, the cooldown timer can be activated if a heater of the device is heated to a minimum threshold. In some example embodiments, the minimum threshold can be at least 60 Joules of energy being injected into the heater. In some example embodiments, the minimum threshold can be the heater being heated for at least ten seconds.

In some example embodiments, the at least one processor can be configured to execute the instructions to cause the cooldown system to set a session underway flag when the session has started and clear the session underway flag when the session has ended.

Also described herein is a non-transitory computer-readable medium including instructions that, when executed by processing circuitry of a device, cause the device to perform the functions described herein. The functions can include detecting when a session has ended, activating a cooldown timer associated with a cooldown time, providing an indication of a remaining time of the cooldown timer, and in response to the cooldown time elapsing, returning the device to normal operation.

Also described herein is a cooldown system for a device. The cooldown system can include a processor means and a memory means. The memory means can be coupled to the processor means and can be configured to store instructions. The processor means can be configured to execute instructions to cause the cooldown system to detect when a session has ended, activate a cooldown timer associated with a cooldown time, provide an indication of a remaining time of the cooldown timer, and in response to the cooldown time elapsing, return the device to normal operation.

Also described herein is a method of operating a cooldown system for a device. The method can include detecting when a session has ended, activating a cooldown timer associated with a cooldown time, providing an indication of a remaining time of the cooldown timer, and in response to the cooldown time elapsing, returning the device to normal operation.

Objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

The various features and advantages of the non-limiting embodiments herein may become more apparent upon review of the detailed description in conjunction with the accompanying drawings. The accompanying drawings are merely provided for illustrative purposes and should not be interpreted to limit the scope of the claims. The accompanying drawings are not to be considered as drawn to scale unless explicitly noted. For purposes of clarity, various dimensions of the drawings may have been exaggerated.

FIG. 1 is a top right, front perspective view of a device in accordance with at least one example embodiment.

FIG. 2A is a top right, front perspective view of the device, where the lid is opened and where the device includes a capsule.

FIG. 2B is a bottom perspective view of the device.

FIG. 2C is a bottom-up view of the device.

FIG. 3 is a block diagram of a cooldown system of the device according to an example embodiment.

FIGS. 4A-4H are different embodiments of icons that may be presented on a communication screen of the device.

FIG. 5 is a method of operating the cooldown system of the device according to an example embodiment.

FIG. 6 is a method of operating the cooldown system of the device according to an example embodiment.

FIG. 7 is a method of analyzing a status of the device when a lid of the device is opened according to an example embodiment.

### DESCRIPTION OF EXAMPLE EMBODIMENTS

Some detailed example embodiments are disclosed herein. However, specific structural and functional details disclosed herein are merely representative for purposes of describing example embodiments. Example embodiments may, however, be embodied in many alternate forms and should not be construed as limited to only the example embodiments set forth herein.

Accordingly, while example embodiments are capable of various modifications and alternative forms, example embodiments thereof are shown by way of example in the drawings and will herein be described in detail. It should be understood, however, that there is no intent to limit example embodiments to the particular forms disclosed, but to the contrary, example embodiments are to cover all modifications, equivalents, and alternatives falling within the scope of example embodiments. Like numbers refer to like elements throughout the description of the figures.

It should be understood that when an element or layer is referred to as being “on,” “connected to,” “coupled to,” or “covering” another element or layer, it may be directly on, connected to, coupled to, or covering the other element or layer or intervening elements or layers may be present. In contrast, when an element is referred to as being “directly on,” “directly connected to,” or “directly coupled to” another element or layer, there are no intervening elements or layers present. As used herein, the term “and/or” includes any and all combinations of one or more of the associated listed items.

It should be understood that, although the terms first, second, third, etc. may be used herein to describe various elements, regions, layers and/or sections, these elements, regions, layers, and/or sections should not be limited by these terms. These terms are only used to distinguish one element, region, layer, or section from another region, layer, or section. Thus, a first element, component, region, layer, or section discussed below could be termed a second element, region, layer, or section without departing from the teachings of example embodiments.

Spatially relative terms (e.g., “beneath,” “below,” “lower,” “above,” “upper,” and the like) may be used herein for ease of description to describe one element or feature's relationship to another element(s) or feature(s) as illustrated in the figures. It should be understood that the spatially relative terms are intended to encompass different orientations of the device in use or operation in addition to the orientation depicted in the figures. For example, if the device in the figures is turned over, elements described as “below,” or “beneath” other elements or features would then be oriented “above” the other elements or features. Thus, the term “below” may encompass both an orientation of above and below. The device may be otherwise oriented (rotated 90 degrees or at other orientations) and the spatially relative descriptors used herein interpreted accordingly.

The terminology used herein is for the purpose of describing various example embodiments only and is not intended to be limiting of example embodiments. As used herein, the singular forms “a,” “an,” and “the” are intended to include the plural forms as well, unless the context clearly indicates otherwise. It will be further understood that the terms “includes,” “including,” “comprises,” and/or “comprising,” specify the presence of stated features, integers, steps, operations, and/or elements, but do not preclude the presence or addition of one or more other features, integers, steps, operations, elements, and/or groups thereof.

When the terms “about” or “substantially” are used in this specification in connection with a numerical value, it is intended that the associated numerical value includes a manufacturing or operational tolerance (e.g., ±10%) around the stated numerical value. Moreover, when the terms “generally” or “substantially” are used in connection with geometric shapes, it is intended that precision of the geometric shape is not required but that latitude for the shape is within the scope of the disclosure. Furthermore, regardless of whether numerical values or shapes are modified as “about,” “generally,” or “substantially,” it will be understood that these values and shapes should be construed as including a manufacturing or operational tolerance (e.g., ±10%) around the stated numerical values or shapes.

Unless otherwise defined, all terms (including technical and scientific terms) used herein have the same meaning as commonly understood by one of ordinary skill in the art to which example embodiments belong. It will be further understood that terms, including those defined in commonly used dictionaries, should be interpreted as having a meaning that is consistent with their meaning in the context of the relevant art and will not be interpreted in an idealized or overly formal sense unless expressly so defined herein.

As used herein, “coupled” includes both removably coupled and permanently coupled. For example, when an elastic layer and a support layer are removably coupled to one another, the elastic layer and the support layer can be separated upon the application of sufficient force.

Hardware may be implemented using processing or control circuitry such as, but not limited to, one or more processors, one or more Central Processing Units (CPUs), one or more microcontrollers, one or more arithmetic logic units (ALUs), one or more digital signal processors (DSPs), one or more microcomputers, one or more field programmable gate arrays (FPGAs), one or more System-on-Chips (SoCs), one or more programmable logic units (PLUs), one or more microprocessors, one or more Application Specific Integrated Circuits (ASICs), or any other device or devices capable of responding to and executing instructions in a defined manner.

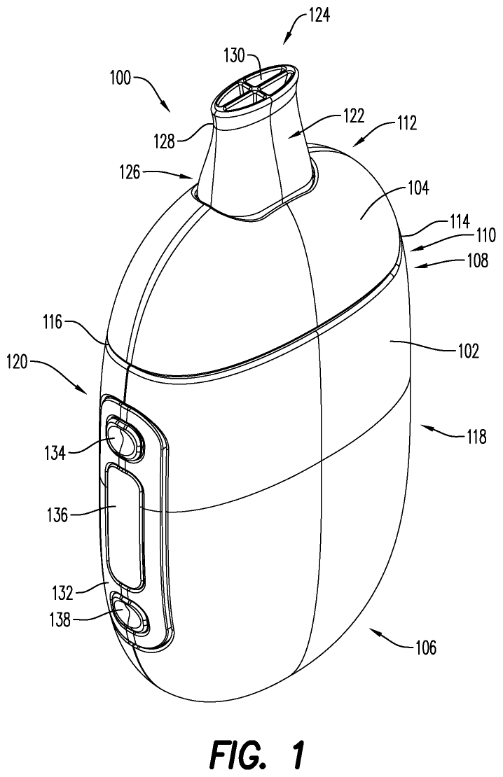

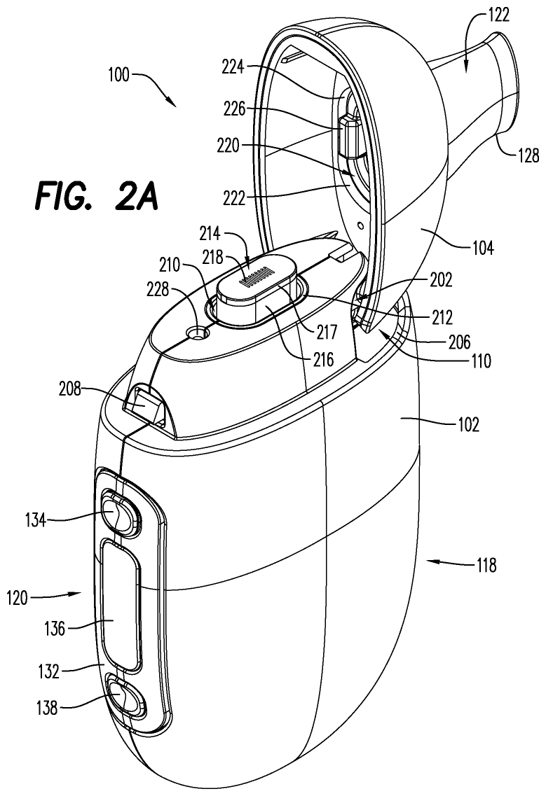

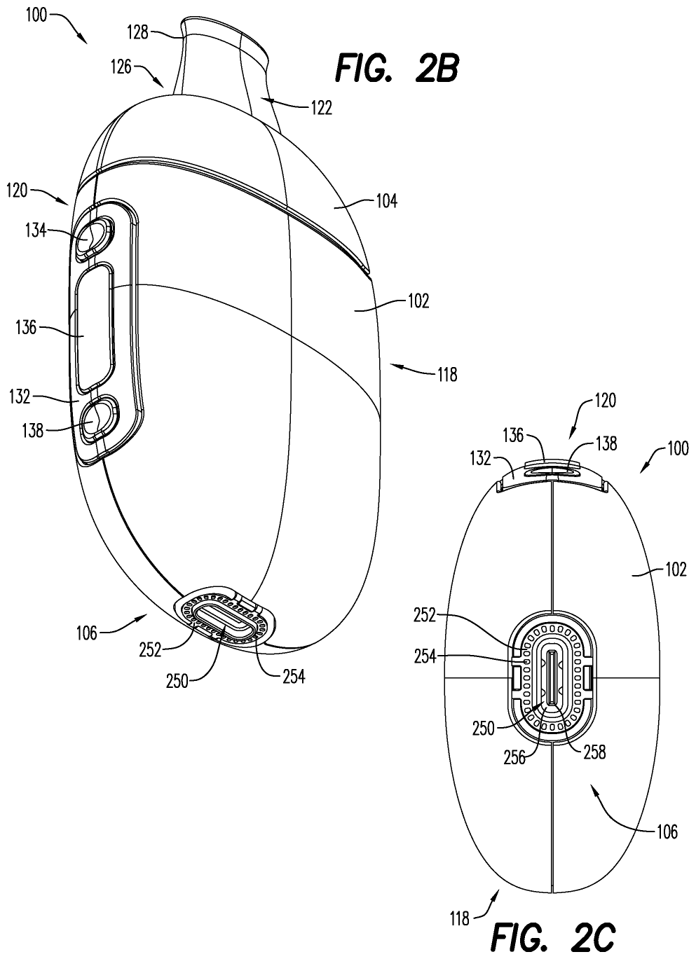

FIGS. 1 and 2A-2C are illustrations of a device 100 according to some example embodiments. In some embodiments, the device 100 may be an aerosol-generating device (e.g., heat-not-burn (HNB) aerosol-generating device). Referring to FIG. 1, a top perspective view of the device 100 is shown. In some embodiments, a main body of the device 100 may have a general oblong or pebble shape. The main body of the device 100 may include a housing 102 and a lid mechanism or a lid 104. The housing 102 may have a first end 106 and a second end 108 opposite the first end 106. The lid may have a first end 110 and a second end 112 opposite the first end 110. The first end 110 of the lid 104 may be fixedly coupled to the second end 108 of the housing 102 at a first point 114 and releasably couplable to the second end 108 of the housing 102 at a second point 116. The first point 114 of the housing 102 may be on a first side 118 of the device 100. The second point 116 of the housing 102 may be on a second side 120 of the device 100.

In some example embodiments, the device 100 may further include a mouthpiece 122. In at least some example embodiments, the mouthpiece 122 may include a first end 124 and a second end 126 opposite the first end 124. The second end 126 of the mouthpiece 122 may be coupled to the second end 112 of the lid 104. In some embodiments, the second end 126 of the mouthpiece 122 may be releasably coupled to the second end 112 of the lid 104. In at least one example embodiment, the mouthpiece 122 may be tapered between the first end 124 and the second end 126. For example, the diameter or average length/width dimensions of the first end 124 may be smaller than the diameter or average length/width dimensions of the second end 126. Towards the first end 124, the taper may have a slight inward curvature 128 that is configured to receive the lips of an adult consumer and improve the comfort and experience. In some embodiments, the first end 124 may have an oblong or elliptical shape and may include one or more outlets 130. For example, the first end 124 may include four outlets 130, such that four or more different areas or quadrants of the adult consumer's mouth can be engaged during use of the device 100. In other embodiments, the mouthpiece 122 may have fewer outlets than the four outlets 130 or more outlets than the four outlets 130.

In some example embodiments, the housing 102 may include a consumer interface panel 132 disposed on the second side 120 of the device 100. For example, the consumer interface panel 132 may be an oval-shaped panel that runs along the second side 120 of the device 100. The consumer interface panel 132 may include a latch release button 134, as well as a communication screen 136 and/or a control button 138. For example, in at least some example embodiments, the consumer interface panel 132 may include the communication screen 136 disposed between the latch release button 134 and the control button 138. As illustrated, the latch release button 134 may be disposed towards the second end 108 of the device 100, and the control button 138 may be disposed towards the first end 106 of the device 100. The latch release button 134 and the control button 138 may be adult consumer interaction buttons. The latch release button 134 and the control button 138 may have a substantially circular shape with a center depression or dimple configured to direct the pressure applied by the adult consumer, although example embodiments are not limited thereto. The control button 138 may turn on and off the device 100. Though only the two buttons are illustrated, it should be understood more or less buttons may be provided depending on the available features and desired adult consumer interface.

The communication screen 136 may be a user interface such as a human-machine interface (HMI) display. In at least one example embodiment, the communication screen 136 may be an integrated thin-film transistor (“TFT”) screen. In other example embodiments, the communication screen 136 is an organic light emitting diode (“OLED”) or light emitting diode (“LED”) screen. The communication screen 136 is configured for adult consumer engagement and may have a generally oblong shape.

In some embodiments, an exterior of the housing 102 and/or the lid 104 may be formed from a metal (such as aluminum, stainless steel, and the like); an aesthetic, food contact rated plastic (such as, a polycarbonate (PC), acrylonitrile butadiene styrene (ABS) material, liquid crystalline polymer (LCP), a copolyester plastic, or any other suitable polymer and/or plastic); or any combination thereof. The mouthpiece 122 may be similarly formed from a metal (such as aluminum, stainless steel, and the like); an aesthetic, food contact rated plastic (such as, a polycarbonate (PC), acrylonitrile butadiene styrene (ABS) material, liquid crystalline polymer (LCP), a copolyester plastic, or any other suitable polymer and/or plastic); and/or plant-based materials (such as wood, bamboo, and the like). One or more interior surfaces or the housing 102 and/or the lid 104 may be formed from or coated with a high temperature plastic (such as, polyetheretherketone (PEEK), liquid crystal polymer (LCP), or the like).

FIG. 2A shows another top perspective view of the device 100 with the lid 104 in an open configuration. The lid 104 may be fixedly coupled to the housing 102 at the first point 114 by a hinge 202, or other similar connector, that allows the lid 104 to move (e.g., swing and rotate) from an open position to a closed position. In some embodiments, the hinge 202 may be a torsion spring. In at least some example embodiments, the housing 102 may include a recess 204 at the first point 114. The recess 204 may be configured to receive a portion of the lid 104 so as to allow for an easy and smooth movement of the lid 104 from the open position to the closed position (and vice versa). The recess 204 may have a structure that corresponds with a relative portion of the lid 104. For example, as illustrated, the recess 204 may include a substantially curved portion 206 that has a general concave shape that corresponds with the curvature of the lid 104, which has a general convex shape.

The lid 104 may be releasably couplable to the housing 102 at the second point 116 by a latch 208, or other similar connector, that allows the lid 104 to be fixed or secured in the closed position and easily releasable to allow the lid 104 to move from the closed position to the open position. In at least one example embodiment, the latch 208 may be coupled to a latch release mechanism disposed within the housing. The latch release mechanism may be configured to move the latch 208 from a first or closed position to a second or open position.

When the lid 104 is in the open position as shown in FIG. 2, a capsule receiving cavity 210 of the housing 102 is exposed. A capsule connector 212 may define the capsule receiving cavity 210 of the housing 102. In some embodiments, the capsule connector 212 may be mounted or otherwise secured to a printed circuit board (PCB) within the housing 102.

As shown in FIG. 2A, a capsule 214 may be received by the capsule receiving cavity 210. In some embodiments, not pictured herein, there may be a gasket disposed around the capsule 214 to help secure the capsule 214 in place within the housing 102. The capsule 214 may include a housing 216 configured to contain an aerosol-forming substrate and a heater. In some embodiments, the housing 216 may be in the form of a cover such as a shell or a box sleeve. In some embodiments, the capsule 214 can include a first end cap 217 and a second end cap. The second end cap may be opposite the first end cap 217 such that is disposed within the housing 102 when the capsule 214 is received by the capsule receiving cavity 210.

As discussed herein, an aerosol-forming substrate is a material or combination of materials that may yield an aerosol. An aerosol relates to the matter generated or output by the devices disclosed, claimed, and equivalents thereof. The material may include a compound (e.g., nicotine, cannabinoid), wherein an aerosol including the compound is produced when the material is heated. The heating may be below the combustion temperature so as to produce an aerosol without involving a substantial pyrolysis of the aerosol-forming substrate or the substantial generation of combustion byproducts (if any). Thus, in an example embodiment, pyrolysis does not occur during the heating and resulting production of aerosol. In other instances, there may be some pyrolysis and combustion byproducts, but the extent may be considered relatively minor and/or merely incidental.

The aerosol-forming substrate may be a fibrous material. For instance, the fibrous material may be a botanical material. The fibrous material is configured to release a compound when heated. The compound may be a naturally occurring constituent of the fibrous material. For instance, the fibrous material may be plant material such as tobacco, and the compound released may be nicotine. The term “tobacco” includes any tobacco plant material including tobacco leaf, tobacco plug, reconstituted tobacco, compressed tobacco, shaped tobacco, or powder tobacco, and combinations thereof from one or more species of tobacco plants, such as Nicotiana rustica and Nicotiana tabacum.

In some example embodiments, the tobacco material may include material from any member of the genus Nicotiana. In addition, the tobacco material may include a blend of two or more different tobacco varieties. Examples of suitable types of tobacco materials that may be used include, but are not limited to, flue-cured tobacco, Burley tobacco, Dark tobacco, Maryland tobacco, Oriental tobacco, rare tobacco, specialty tobacco, blends thereof, and the like. The tobacco material may be provided in any suitable form, including, but not limited to, tobacco lamina, processed tobacco materials, such as volume expanded or puffed tobacco, processed tobacco stems, such as cut-rolled or cut-puffed stems, reconstituted tobacco materials, blends thereof, and the like. In some example embodiments, the tobacco material is in the form of a substantially dry tobacco mass. Furthermore, in some instances, the tobacco material may be mixed and/or combined with at least one of propylene glycol, glycerin, sub-combinations thereof, or combinations thereof.

The compound may also be a naturally occurring constituent of a medicinal plant that has a medically-accepted therapeutic effect. For instance, the medicinal plant may be a cannabis plant, and the compound may be a cannabinoid. Cannabinoids interact with receptors in the body to produce a wide range of effects. As a result, cannabinoids have been used for a variety of medicinal purposes (e.g., treatment of pain, nausea, epilepsy, psychiatric disorders). The fibrous material may include the leaf and/or flower material from one or more species of cannabis plants such as Cannabis sativa, Cannabis indica, and Cannabis ruderalis. In some instances, the fibrous material is a mixture of 60-80% (e.g., 70%) Cannabis sativa and 20-40% (e.g., 30%) Cannabis indica.

Examples of cannabinoids include tetrahydrocannabinolic acid (THCA), tetrahydrocannabinol (THC), cannabidiolic acid (CBDA), cannabidiol (CBD), cannabinol (CBN), cannabicyclol (CBL), cannabichromene (CBC), and cannabigerol (CBG). Tetrahydrocannabinolic acid (THCA) is a precursor of tetrahydrocannabinol (THC), while cannabidiolic acid (CBDA) is precursor of cannabidiol (CBD). Tetrahydrocannabinolic acid (THCA) and cannabidiolic acid (CBDA) may be converted to tetrahydrocannabinol (THC) and cannabidiol (CBD), respectively, via heating. In an example embodiment, heat from a heater may cause decarboxylation so as to convert the tetrahydrocannabinolic acid (THCA) in the capsule to tetrahydrocannabinol (THC), and/or to convert the cannabidiolic acid (CBDA) in the capsule to cannabidiol (CBD).

In instances where both tetrahydrocannabinolic acid (THCA) and tetrahydrocannabinol (THC) are present in the capsule, the decarboxylation and resulting conversion will cause a decrease in tetrahydrocannabinolic acid (THCA) and an increase in tetrahydrocannabinol (THC). At least 50% (e.g., at least 87%) of the tetrahydrocannabinolic acid (THCA) may be converted to tetrahydrocannabinol (THC) during the heating of the capsule. Similarly, in instances where both cannabidiolic acid (CBDA) and cannabidiol (CBD) are present in the capsule, the decarboxylation and resulting conversion will cause a decrease in cannabidiolic acid (CBDA) and an increase in cannabidiol (CBD). At least 50% (e.g., at least 87%) of the cannabidiolic acid (CBDA) may be converted to cannabidiol (CBD) during the heating of the capsule.

Furthermore, the compound may be or may additionally include a non-naturally occurring additive that is subsequently introduced into the fibrous material. In one instance, the fibrous material may include at least one of cotton, polyethylene, polyester, rayon, combinations thereof, or the like (e.g., in a form of a gauze). In another instance, the fibrous material may be a cellulose material (e.g., non-tobacco and/or non-cannabis material). In either instance, the compound introduced may include nicotine, cannabinoids, and/or flavorants. The flavorants may be from natural sources, such as plant extracts (e.g., tobacco extract, cannabis extract), and/or artificial sources. In yet another instance, when the fibrous material includes tobacco and/or cannabis, the compound may be or may additionally include one or more flavorants (e.g., menthol, mint, vanilla). Thus, the compound within the aerosol-forming substrate may include naturally occurring constituents and/or non-naturally occurring additives. In this regard, it should be understood that existing levels of the naturally occurring constituents of the aerosol-forming substrate may be increased through supplementation. For example, the existing levels of nicotine in a quantity of tobacco may be increased through supplementation with an extract containing nicotine. Similarly, the existing levels of one or more cannabinoids in a quantity of cannabis may be increased through supplementation with an extract containing such cannabinoids.

The first end cap 217 can include a first opening 218. In some embodiments, the first opening 218 may be a series of openings disposed through the first end cap 217. Similarly, the second end cap can include a second opening that may be a series of openings in some embodiments. In some embodiments, the first end cap 217 and/or the second end cap may be transparent so as to serve as windows configured to permit a viewing of the contents/components (e.g., aerosol-forming substrate and/or heater) within the capsule 214.

The capsule receiving cavity 210 may have a base that may be inside the housing 102. In some embodiments, the base may include at least one contact point that may be configured to couple to one or more contact points of the capsule 214 when the capsule 214 is received by the capsule receiving cavity 210. When the capsule 214 is inserted into the capsule receiving cavity 210, the weight of the capsule 214 itself may not be sufficient to compress the at least one contact point of the base of the capsule receiving cavity 210. As a result, the capsule 214 may simply rest on exposed pins of the at least one contact point without any compression (or without any significant compression) of electrical contacts of the at least one contact point. Additionally, the weight of the lid 104 itself, when pivoted to transition to a closed position, may not compress the electrical contacts of the at least one contact point to any significant degree and, instead, may simply rest on the capsule 214 in an intermediate, partially open/closed position. In such an instance, a deliberate action (e.g., downward force) to close the lid 104 will cause a surface 220 of the lid 104 to press down onto the capsule 214 to provide the desired seal and also cause the capsule 214 to compress and, thus, fully engage the electrical contacts of the at least one contact point.

Additionally, a full closure of the lid 104 may result in an engagement with the latch 208, which may maintain the closed position and the desired mechanical/electrical engagements involving the capsule 214 until released (e.g., via the latch release button 134). The force requirement for closing the lid 104 may help to ensure and/or improve air/aerosol sealing and to provide a more robust electrical connection, as well as improved device and thermal efficiency and battery life by reducing or eliminating early power draws and/or parasitic heating of the capsule 214.

The lid 104 may include an inner cavity 222 that may be adapted to receive the housing 102 when the lid is in the closed position. In some embodiments, the inner cavity 222 of the lid 104 may include an impingement or engagement member or the surface 220 configured to engage the capsule 214 when the lid 104 is pivoted to transition to the closed position. The surface 220 of the lid 104 may include a recess that may correspond to the size and shape of the capsule and/or a resilient material to enhance an interface with the capsule to provide the desired seal. In some embodiments, the lid 104 may further include an opening 224 that may be adapted to receive the second end 126 of the mouthpiece 122. The mouthpiece 122 may include at least one extension 226 that may be received by the opening 224 of the lid 104 to secure the mouthpiece 122 to the lid 104. In some embodiments, the lid 104 may further include a projection that may be configured to couple with a recess 228 of the housing 102. The projection may fit within the recess 228 when the lid 104 is coupled to the housing 102 in the closed position.

Referring to FIG. 2B, a bottom perspective view of the device 100 is shown. In some embodiments, the housing 102 may define a port or a charging connector 250. The charging connector may be defined or disposed the first end 106 of the housing 102. The charging connector 250 may be configured to receive an electric current (e.g., via a USB/mini-USB cable) from an external power source so as to charge a power source internal to the device 100. In some embodiments, a protective grille 252 is disposed around the charging connector 250. The protective grille 252 may be configured to help reduce or prevent debris ingress and/or the inadvertent blockage of the incoming airflow. For example, the protective grille 252 may define a plurality of pores 254 along its length or course. As illustrated, the protective grille 252 may have an annular form that surrounds the charging connector 250. In this regard, the pores 254 may also be arranged (e.g., in a serial arrangement) around the charging connector 250. Each of the pores 254 may have an oval or circular shape, although not limited thereto. In at least one example embodiment, the protective grille 252 may include an approved food contact material. For example, the protective grille 252 may include plastic, metal (e.g., stainless steel, aluminum), or any combination thereof. In at least one example embodiment, a surface of the protective grille 252 may be coated, for example with a thin layer of plastic, and/or anodized.

The pores 254 in the protective grille 252 may function as inlets for air drawn into the device 100. During the operation of the device 100, ambient air entering through the pores 254 in the protective grille 252 around the charging connector 250 will converge to form a combined flow that then travels to the capsule 214. For example, the pores 254 may be in fluidic communication with the capsule receiving cavity 210. In at least one example embodiment, air may be drawn from the pores 254 and through the capsule receiving cavity 210. For example, air may be drawn through the capsule 214 received by the capsule receiving cavity 210 and out of the mouthpiece 122.

Referring to FIG. 2C, a bottom-up view of the device 100 is shown. In some embodiments, the charging connector 250 may be an assembly defining a cavity 256 that has a projection 258 within the cavity 256. In at least one example embodiment, the projection 258 does not extend beyond the rim of the cavity 256. In addition, the charging connector 250 may also be configured to send data to and/or receive data (e.g., via a USB/mini-USB cable) from another aerosol generating device (e.g., heat not-burn (HNB) aerosol generating device) and/or other electronic device (e.g., phone, tablet, computer, and the like). In at least one embodiment, the device 100 may instead or additionally be configured for wireless communication (e.g., via Bluetooth) with such other aerosol generating devices and/or electronic devices.

As should be understood, the device 100 and capsule 214 include additional components (e.g., heater and internal air flow path) such as described in Atty. Docket No. 24000NV-000847-US, entitled “HEAT-NOT-BURN (HNB) AEROSOL-GENERATING DEVICES AND CAPSULES”, filed on the same day herewith and assigned application Ser. No. ______, the entire contents of which are herein incorporated by reference.

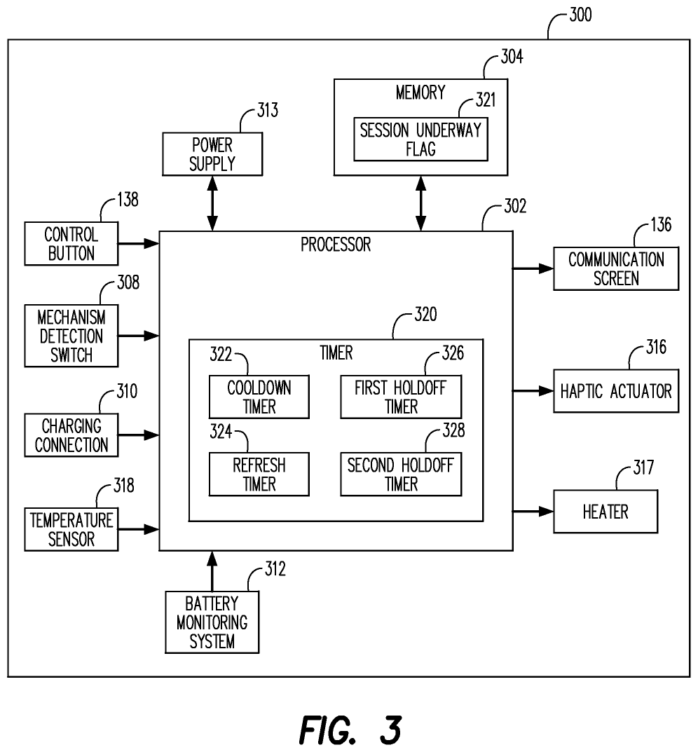

Referring to FIG. 3, a block diagram of a cooldown system 300 of the device 100 according to an example embodiment is shown. The cooldown system 300 may be used to alert or inform a consumer that the device 100 is warm after heating of a consumable such as the capsule 214 of the device 100. More specifically, the cooldown system 300 may inform the consumer of how long it will be before the capsule 214 of the device 100 can be removed and the point at which the capsule 214 may be removed. Additionally, the cooldown system 300 may be configured to alert the consumer that the capsule 214 is warm if the lid 104 of the device 100 is opened prior to the capsule 214 cooling to a comfortable or desired temperature. The cooldown system 300 may ensure that the device 100 is not turned off automatically until the cooldown system 300 has alerted the consumer that the device 100 is in a state where the lid 104 may be opened to remove the capsule 214 of the device 100.

The cooldown system 300 may include a processor 302, a memory 304, the control button 138, a mechanism detection switch 308, a charging connection 310, a battery monitoring system 312, a power supply 313, the communication screen 136, a haptic actuator 316, a heater 317, and a temperature sensor 318. In some embodiments, the processor 302 may include a timer 320 and the memory 304 may include a session underway flag 321. In other embodiments, the session underway flag 321 may be stored in the processor 302 such as in local storage of the processor 302 and the timer 320 may be executed using instructions stored in the memory 304. The processor 302 may communicate with the memory 304, the control button 138, the mechanism detection switch 308, the charging connection 310, the battery monitoring system 312, the power supply 313, the communication screen 136, the haptic actuator 316, the heater 317, the temperature sensor 318, and the timer 320.

The processor 302 may be hardware including logic circuits, a hardware/software combination that may be configured to execute software, or a combination thereof. For example, the processor 302 may include, but is not limited to, a central processing unit (CPU), an arithmetic logic unit (ALU), a digital signal processor, a microcomputer, a field programmable gate array (FPGA), a System-on-Chip (SoC), a programmable logic unit, a microprocessor, application-specific integrated circuit (ASIC), or another similar device. The processor 302 may be configured as a special purpose machine (e.g., a processing device) to execute the software or instructions, stored in the memory 304. The software may be embodied as program code including instructions for performing and/or controlling any or all operations described herein as being performed by the processor 302.

In other example embodiments, other processing circuitry and/or control circuitry may be used.

The memory 304 is illustrated as being external to the processor 302, in some example embodiments the memory 304 may be on board the processor 302. The memory 304 may describe any of the terms “storage medium”, “computer readable storage medium” or “non-transitory computer readable storage medium” and may represent one or more devices for storing data, including read only memory (ROM), random access memory (RAM), magnetic RAM, core memory, magnetic disk storage mediums, optical storage mediums, flash memory devices and/or other tangible machine-readable mediums for storing information. The term “computer-readable medium” may include, but is not limited to, portable or fixed storage devices, optical storage devices, and various other mediums capable of storing, containing, or carrying instructions and/or data.

The session underway flag 321 may be stored in the memory 304 and may be set to indicate a status of a session of the device 100. For example, the session underway flag 321 may be set by the processor 302 when the control button 138 is pressed to start a session of the device 100. The session underway flag 321 may be reset by the processor 302 when a session of the device 100 has ended such as when the control button 138 is pressed to end the session of the device 100.

The timer 320 may include one or more timers configured to measure one or more times related to the device 100 and/or the cooldown system 300. The timer 320 may include a cooldown timer 322 that may be configured to measure a cooldown time. The cooldown time may be a length of time from an end of a session of use of the device 100 until the device 100 is cooled to a temperature where the capsule 214 can be removed from the device 100. The timer 320 may include a refresh timer 324 that may be configured to measure a refresh time. The refresh time may be less than the cooldown time in some embodiments. When the refresh time elapses, information related to the cooldown time may be recalculated and refreshed on the communication screen 136. The timer 320 may include a first holdoff timer 326 that may measure a first holdoff time. The first holdoff time may be an amount of time that the communication screen 136 may display a first icon while the device 100 is powering off prior to displaying any information related to the cooldown time. The timer 320 may additionally include a second holdoff timer 328 that may be configured to measure a second holdoff time. The second holdoff time may be an amount of time that the communication screen 136 may display a power off indication if the device 100 is turned off while the cooldown timer 322 is active.

The control button 138 may be configured to generate a signal indicating that a consumer has switched the device 100 to an “off” state to end an ongoing session of the device 100.

The mechanism detection switch 308 may be configured to generate a signal indicating that a lid mechanism such as the lid 104 of the device 100 has been opened. The mechanism detection switch 308 may be a push button switch, a toggle button, a capacitive sensor, an IR sensor, a magnetic detection sensor such as a Hall Effect sensor, or another element that is configured to communicate with the processor 302 that the lid 104 of the device 100 has been opened. When the lid 104 of the device 100 is opened, any ongoing session of the device 100 may end. The mechanism detection switch 308 may be configured to generate the signal indicating that the lid 104 has been opened when the latch release mechanism of the device 100 releases the latch 208. Additionally or alternatively, the mechanism detection switch 308 may be coupled to the latch release button 134 and may generate the signal indicating that the lid 104 of the device 100 has been opened when the latch release button 134 is pressed. More specifically, the mechanism detection switch 308 may be located within the recess 228 such that closing the lid 104 of the device 100 actuates the mechanism detection switch 308.

The charging connection 310 may be configured to generate a signal indicating that the device 100 is connected to a charger. When the device 100 is connected to a charger, any ongoing sessions may end. In some embodiments, the housing 102 of the device 100 may include a charging connector or port. For example, the port may be defined/disposed in the first end 106 of the housing 102. The port may be configured to receive an electric current (e.g., via a USB/mini-USB cable) from an external power source to charge a power source such as the power supply 313 internal to the device 100. The charging connection 310 may be configured to detect when the port of the device 100 is receiving an electric current which may also indicate that any ongoing session has ended.

The battery monitoring system 312 may be configured to generate a signal indicating that the device 100 has entered a low battery state. In some embodiments, when the device 100 enters a low battery state, any ongoing sessions may end.

In some embodiments, the housing 102 of the device 100 may enclose or house the power supply 313. The power supply 313 may include one or more batteries such as a rechargeable dual battery arrangement, a lithium-ion battery, and/or fuel cells. The power supply 313 may be configured to receive the electric current supplied to the device 100 through the port to charge the power supply 313. The battery monitoring system 312 may generate a signal indicating that the device 100 has entered a low battery state when the power supply 313 reaches a charge that is lower than a predetermined threshold. When the device 100 is in a low battery state, the device 100 may not have enough power to continue normal operation of a session. However, the device 100 may have enough power to operate the cooldown system 300 until the capsule 214 is at a comfortable temperature for a consumer to remove.

The communication screen 136 may display information related to the device 100. The communication screen 136 may display one or more icons to communicate information related to the device 100. For example, the communication screen 136 may display a timer icon that may indicate a remaining time of the cooldown timer 322 of the device 100. The communication screen 136 may also display an alert icon that may indicate that the lid 104 of the device 100 has been opened while the cooldown timer 322 is active. The communication screen 136 may also be configured to display a cooldown alert icon that may indicate that the lid 104 of the device has been opened while a session is underway and prior to the cooldown timer 322 being activated. The communication screen 136 may also be configured to display a capsule eject icon that may indicate when the cooldown time measured by the cooldown timer 322 has elapsed. The communication screen 136 may also be configured to display a session end icon that may indicate that the device 100 is being powered off while the cooldown timer 322 is being activated to measure the cooldown time. The communication screen 136 may also be configured to display a first power off icon and a second power off icon that may both indicate that the control button 138 has been pressed while the cooldown timer 322 is active to power the device off. The communication screen 136 may also be configured to display a cooldown aware icon that may indicate that the cooldown system 300 is still operating after the consumer has pressed the control button 138 to power off the device 100. The communication screen 136 may also be configured to display a fault icon that may indicate that the device 100 is in a fault state and may not be able to operate as intended.

The haptic actuator 316 may be a haptic motor that may be disposed within the housing 102 of the device 100. The haptic actuator 316 may be configured to vibrate the device when the haptic actuator 316 is actuated. The haptic actuator 316 may be configured to be actuated by the processor 302 when a consumer opens the lid 104 of the device 100 while the timer 320 is activated to measure the cooldown time. In some embodiments, the haptic actuator 316 may be configured to vibrate the device 100 in a vibration pattern when actuated. For example, the haptic actuator 316 may be configured to vibrate the device 100 three times when the lid 104 of the device 100 is opened while the timer 320 is activated to measure the cooldown time. The vibration pattern may be unique to the cooldown system 300 such that a consumer is able to distinguish the vibration pattern from other vibrations that the haptic actuator 316 may perform during operation of the device 100 other than when the cooldown system 300 is operational.

The heater 317 may be housed within the device 100 and may be configured to heat the capsule 214 of the device 100. In some embodiments, the heater 317 may be an element of or may be coupled to one or more of a heating voltage measurement circuit, a heating current measurement circuit, and/or a compensation measurement circuit substantially as described in U.S. application Ser. No. 17/151,409 titled “HEAT-NOT-BURN (HNB) AEROSOL-GENERATING DEVICES INCLUDING INTRA-DRAW HEATER CONTROL, AND METHODS OF CONTROLLING A HEATER” filed on Jan. 18, 2021, the disclosure of which is incorporated herein in its entirety by reference.

The temperature sensor 318 may be configured to measure a temperature of the device 100. In some embodiments, the temperature sensor 318 may be a may be a thermistor or thermocouple. More specifically, the temperature sensor 318 may be disposed proximate to the capsule 214 of the device 100 and may be configured to measure a temperature of the of an area proximate to the capsule 214 of the device 100 to determine when the capsule 214 may be removed from the device 100. In some embodiments, the cooldown time may be determined based on a temperature measured by the temperature sensor 318. For example, the cooldown time may be related to the temperature measured by the temperature sensor 318 such that the cooldown time is longer when the temperature measured by the temperature sensor 318 is higher and is shorter when the temperature measured by the temperature sensor 318 is lower.

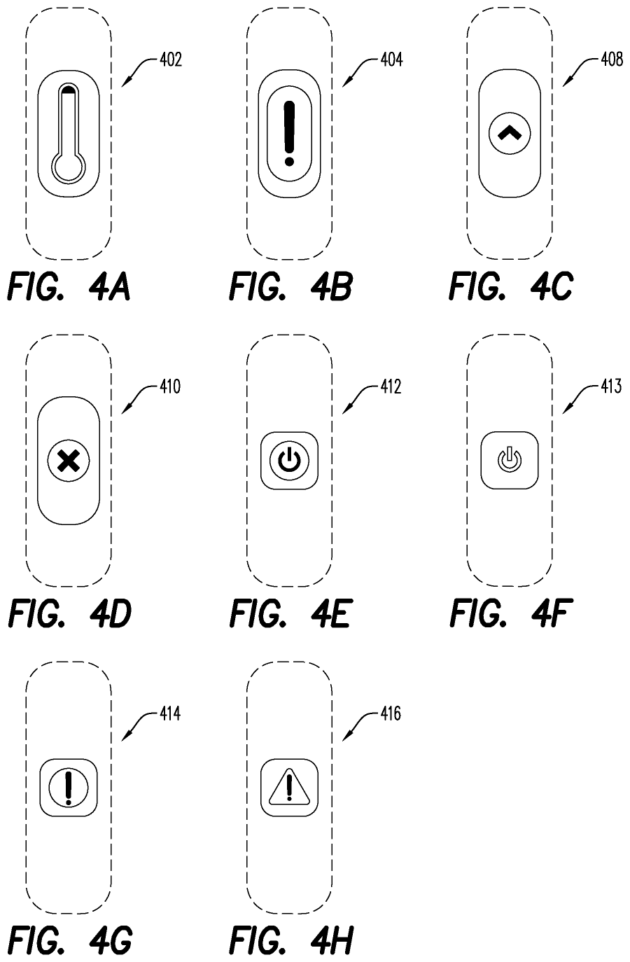

Referring to FIGS. 4A-4H, different example embodiments of display screens having a graphical user interface with an icon are shown. In some embodiments, the display screens may the communication screen 136 of the device 100. The icons displayed on the display screen or the communication screen 136 may generally be referred to as system icons. In some embodiments, the icons of FIGS. 4A-4H may be displayed on the communication screen 136 in a variety of colors, shade, or sizes. For example, the shaded areas of FIGS. 4A-4H may be displayed as orange, teal, or red, or any other suitable color. The broken lines shown in FIGS. 4A-4H illustrate a display screen or portions thereof. Additionally or alternatively, each of the icons of FIGS. 4A-4H may be modified or adapted to be a different symbol or shape but each icon may represent a unique message about the device 100 and/or the cooldown system 300.

FIG. 4A shows a display screen having a graphical user interface with an icon such as a timer icon 402. The timer icon 402 may indicate to a consumer how much time is remaining on the cooldown timer 322 so that the consumer knows when the capsule 214 can be removed from the device 100. The timer icon 402 may be refreshed an updated during the cooldown time to keep the consumer informed about when the capsule 214 will be removable from the device 100. For example, if there is 90% of the cooldown time remaining on the cooldown timer 322, the timer icon 402 may appear substantially as shown in FIG. 4A with approximately 90% of the thermometer filled in or shaded. As the cooldown time elapses, the timer icon 402 may be updated such that a shaded portion of the thermometer corresponds to the cooldown time remaining on the cooldown timer 322.

FIG. 4B shows a display screen having a graphical user interface with an icon such as an alert icon 404. The alert icon 404 may indicate to the consumer that the lid 104 of the device 100 has been opened while the cooldown timer 322 is active or while a session of the device 100 is active. The alert icon 404 may indicate to the consumer that the capsule 214 is not cooled to a comfortable temperature to be removed from the device 100.

FIG. 4C shows a display screen having a graphical user interface with an icon such as a capsule eject icon 408. The capsule eject icon 408 may indicate to the consumer that the cooldown timer 322 has elapsed and the capsule 214 may be removed from the device 100.

FIG. 4D shows a display screen having a graphical user interface with an icon such as a session end icon 410. In some embodiments, the session end icon may indicate that the capsule 214 is empty of the aerosol-forming substrate. The session end icon 410 may indicate that a previously active session of the device 100 has ended. In some embodiments, the session end icon 410 may be briefly displayed on the communication screen 136 prior to the timer icon 402 being displayed.

FIG. 4E shows a display screen having a graphical user interface with an icon such as first power off icon 412. The first power off icon 412 may indicate that the control button 138 has been pressed and held for at least three seconds while the cooldown timer 322 is active. When the control button 138 is pressed for at least three seconds, the device 100 may be powered off after the cooldown timer 322 elapses. The first power off icon 412 may be displayed while the control button 138 is being pressed and while the second holdoff timer 328 is active.

FIG. 4F shows a display screen having a graphical user interface with an icon such as a second power off icon 413. The second power off icon 413 may be displayed after the control button 138 is released and tapped for a second time to confirm that the device 100 should be powered off. Once the control button 138 has been pressed for a second time, the first power off icon 412 is removed from the communication screen 136 by the processor 302. The second power off icon 413 may be similar to the first power off icon 412 but the portion of the icon that is colored may be inversed from the first power off icon 412. The second power off icon 413 may be displayed while the second holdoff timer 328 is active and may be removed from the communication screen 136 when the second holdoff timer elapses.

FIG. 4G shows a display screen having a graphical user interface with an icon such as a cooldown aware icon 414. The cooldown aware icon 414 may indicate that the cooldown system 300 is still operating after the consumer has pressed the control button 138 to power off the device 100. More specifically, the cooldown aware icon 414 may be displayed on the communication screen 136 after the second holdoff timer has elapsed and the first power off icon 412 and the second power off icon 413 have been removed from the communication screen 136. The cooldown aware icon 414 may be displayed until the cooldown timer 322 elapses.

FIG. 4H shows a display screen having a graphical user interface with an icon such as a fault icon 416. The fault icon 416 may indicate that the device 100 is in a non-operational state such that a session cannot begin. In some embodiments, the device may be in a non-operational state or a fault state when the lid 104 of the device 100 has been shut after previously being opened while a session was active or while the cooldown timer 322 was active. In some embodiments, closing the lid 104 after opening the lid 104 during an active session or while the cooldown timer 322 was active may cause a resistance across electrodes of the capsule 214 to be outside of a valid range and may force the device 100 into a fault state. In other embodiments, the device 100 may enter into a fault state if the hardware or the software of the device 100 malfunctions while a session is ongoing or while the cooldown timer 322 is active. If the device 100 enters into a fault state due to a malfunction, any ongoing sessions may be terminated, the cooldown timer 322 may be started, and the fault icon 416 may be displayed on the communication screen 136 by the processor 302. The cooldown system 300 may continue to operate the cooldown timer 322 while the fault icon 416 is shown on the communication screen 136. The fault icon 416 may be displayed on the communication screen 136 until the fault is corrected. If the fault is corrected, the cooldown system 300 may display the appropriate icon of FIGS. 4A to 4G to indicate the state of the cooldown system 300.

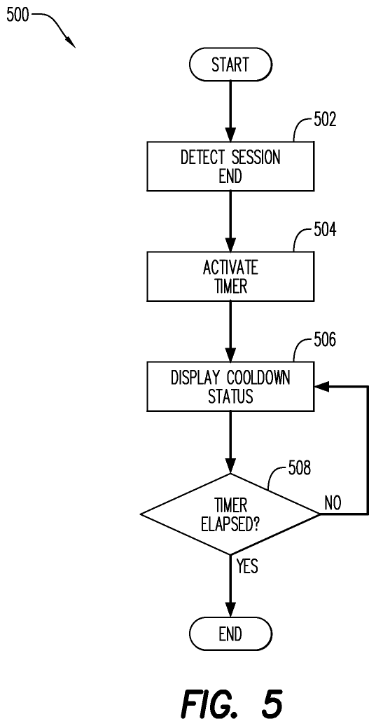

Referring to FIG. 5, a method 500 of operating the cooldown system 300 of the device 100 is shown. The processor 302 is configured to cause the cooldown system 300 to perform the method of FIG. 5 by executing instructions stored by the memory 304. In other example embodiments, the processor 302 may be an ASIC and is configured to cause the cooldown system 300 to perform the method of FIG. 5. The method 500 may start while a session of the device 100 is ongoing. A session may begin when the device 100 is switched on and preheated such that the device 100 may be used by a consumer. In some embodiments, a session may start when the control button 138 is pressed to power the device 100 on. The device 100 may be operating an ongoing session when the capsule 214 is heated such that a consumer may be able to use the device 100. The method 500 may proceed to step 502 where the cooldown system 300 may detect that the session has ended.

The cooldown system 300 may detect that the session has ended if the cooldown system 300 receives a signal from the control button 138 indicating that a consumer has switched the device 100 off (e.g., the consumer presses the control button 138). Additionally or alternatively, the cooldown system 300 may detect that the session has ended if the cooldown system 300 receives a signal from the mechanism detection switch 308 indicating that the lid 104 of the device has been opened. Additionally or alternatively, the cooldown system 300 may detect that the session has ended if the cooldown system 300 receives a signal from the charging connection 310 indicating that the device 100 has been connected to a charger. Additionally or alternatively, the cooldown system 300 may detect that the session has ended if the cooldown system 300 receives a signal from the battery monitoring system 312 indicating that the device 100 has entered a low battery state. Additionally or alternatively, the cooldown system 300 may detect that the session has ended if the cooldown system 300 detects that the device 100 is in a fault state. Additionally or alternatively, the cooldown system 300 may detect that the session has ended if the cooldown system 300 receives a signal from a session control system of the device 100 indicating that a session threshold has been met. In some embodiments, a session threshold may be met if a session has exceeded a time threshold and/or a puff threshold.

As should be understood, the session control system includes additional components and functions such as described in Atty. Docket No. 24000NV-000859-US, entitled “SESSION CONTROL SYSTEM”, filed on the same day herewith and assigned application Ser. No. ______, the entire contents of which are herein incorporated by reference.

After the cooldown system 300 has detected that the session has ended at the step 502, the method may move to step 504. At the step 504, the cooldown system 300 may activate the timer 320. The timer 320 may be the cooldown timer 322 and the cooldown timer 322 may be configured to measure the cooldown time. As described above with reference to FIG. 3, the cooldown time may be a length of time from when the session has ended until the device 100 is cooled to a temperature where the capsule 214 can be removed from the device 100.

In some embodiments, the cooldown time may be about 120 seconds. In other embodiments, the cooldown time may be based on a length of the session that just ended. In some embodiments, the longer the session, the longer the cooldown time. For example, if the session was less than ten seconds, the cooldown time may be thirty seconds. If the session is longer than ten seconds, the cooldown time may be 120 seconds or may be a longer time based on the length of the session.

Additionally or alternatively, the cooldown time may be based on a temperature as measured by the temperature sensor 318 of the device 100.

Additionally or alternatively, the cooldown system 300 may only activate the cooldown timer 322 if the heater 317 of the device 100 is heated to a minimum threshold. In some embodiments, the minimum threshold may be when the heater 317 has been heated for at least a certain amount of time. For example, if the heater 317 has been heated for at least ten seconds, the cooldown system 300 may activate the cooldown timer 322 when a session has ended. In other embodiments, the minimum threshold may be when a threshold amount of energy has been injected into the heater 317. For example, the cooldown system 300 may activate the cooldown timer 322 when at least 60 Joules of energy have been injected into the heater 317.

After the timer 320 is activated at the step 504, the method 500 may proceed to step 506 where a cooldown status is displayed on the communication screen 136. In some embodiments, the communication screen 136 may display the timer icon 402 to indicate a remaining time being measured by the timer 320. More specifically, the timer icon 402 may be configured to indicate an amount of time remaining until the cooldown time as measured by the cooldown timer 322 reaches zero.

After the cooldown status is displayed on the communication screen 136 at the step 506, the method 500 may proceed to conditional step 508 where the cooldown system 300 determines if the timer 320 has elapsed. If the timer 320 has not elapsed, the method 500 may return to the step 506 and may continue to display the cooldown status of the device 100 on the communication screen 136. If the timer 320 has elapsed, the method 500 may proceed to an end of the method 500.

When the method 500 ends, the device 100 may return to normal operation. When the method 500 ends, the device 100 may have a temperature that is low enough to allow a consumer to comfortably remove the capsule 214. In some embodiments, normal operation may allow a consumer to remove the capsule 214 to replace or refill the capsule 214 in order to continue using the device 100.

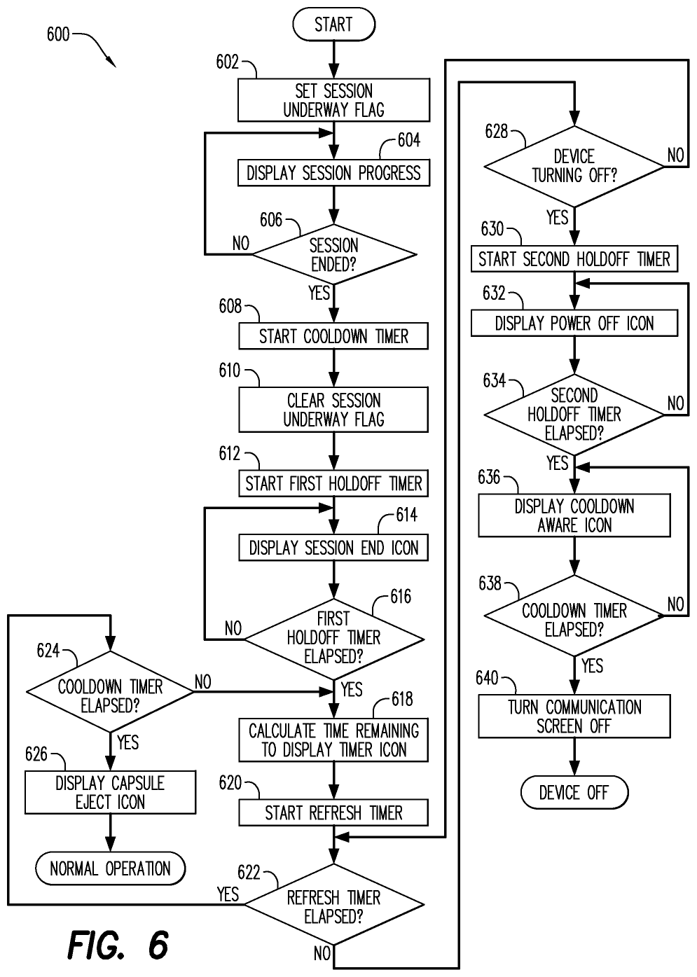

Referring to FIG. 6, a block diagram of a method 600 of operating the cooldown system 300 of the device 100 is shown. The method 600 is more detailed than the method 500 as shown in FIG. 5. The method 600 may start when a session of the device 100 is started. Once the session is started, the method 600 may proceed to step 602 where the session underway flag 321 is set by the processor 302 and/or the memory 304. The session underway flag 321 may indicate that a session is ongoing.

The method 600 may then proceed to step 604 where the communication screen 136 may display a session progress indication or icon. In some embodiments, the session progress indication may inform a consumer of a total amount of time that the session has been in progress. In other embodiments, the session progress indication may be configured to indicate a remaining battery life of the device 100 or how much longer the session may be able to continue based on an amount of material contained in the capsule 214. In still other embodiments, the session progress indication may be another indicator of a state of the session of the device 100.

The method 600 may then progress to conditional step 606 where the cooldown system 300 determines if the session has ended. As described above, the cooldown system 300 may detect that the session has ended if the cooldown system 300 receives a signal from the control button 138 indicating that a consumer has switched the device 100 off. Additionally or alternatively, the cooldown system 300 may detect that the session has ended if the cooldown system 300 receives a signal from the mechanism detection switch 308 indicating that the lid 104 of the device has been opened. Additionally or alternatively, the cooldown system 300 may detect that the session has ended if the cooldown system 300 receives a signal from the charging connection 310 indicating that the device 100 has been connected to a charger. Additionally or alternatively, the cooldown system 300 may detect that the session has ended if the cooldown system 300 receives a signal from the battery monitoring system 312 indicating that the device 100 has entered a low battery state. If the device 100 has entered a low battery state, the device 100 may have enough battery to complete the method 600 and may ensure that the consumer is alerted when the device 100 is in a state where the capsule 214 may be removed from the device 100. Additionally or alternatively, the cooldown system 300 may detect that the session has ended if the cooldown system 300 detects that the device 100 is in a fault state. Additionally or alternatively, the cooldown system 300 may detect that the session has ended if the cooldown system 300 receives a signal from a session control system of the device 100 indicating that a session threshold has been met.

If the session has not ended, the method 600 may return to the step 604 and continue to display the session progress indication. If the session has ended, the method 600 may proceed to step 608.

At the step 608, the cooldown system 300 may start the timer 320. The timer 320 may be the cooldown timer 322 and the cooldown timer 322 may be configured to measure the cooldown time. As described above with reference to FIG. 3, the cooldown time may be a length of time from when the session has ended until the device 100 is cooled to a temperature where the capsule 214 can be removed from the device 100.

Additionally or alternatively, the cooldown time may be based on a temperature as measured by the temperature sensor 318 of the device 100.

Additionally or alternatively, the cooldown system 300 may only activate the cooldown timer 322 if the heater 317 of the device 100 is heated to a minimum threshold. In some embodiments, the minimum threshold may be when the heater 317 has been heated for at least a certain amount of time. For example, if the heater 317 has been heated for at least ten seconds, the cooldown system 300 may activate the cooldown timer 322 when a session has ended. In other embodiments, the minimum threshold may be when a threshold amount of energy has been injected into the heater 317. For example, the cooldown system 300 may activate the cooldown timer 322 when at least 60 Joules of energy have been injected into the heater 317.

The method 600 may proceed to step 610 after the timer 320 is activated at the step 608. At the step 610, the processor 302 and/or the memory 304 of the cooldown system 300 may clear the session underway flag 321 that was set at the step 602. The session underway flag 321 may be cleared because the cooldown system 300 has determined that the session has ended.

The method 600 may proceed to step 612 after the session underway flag 321 is cleared at the step 610. At the step 612, the first holdoff timer 326 of the timer 320 may be started by the processor 302. The first holdoff timer 326 may be configured to measure a first holdoff time. In some embodiments, the first holdoff time may be an amount of time that indications or information is displayed on the communication screen 136 prior to any information related to the cooldown time. In some embodiments, the first holdoff time may be about three seconds.

The method 600 may proceed to step 614 after the holdoff timer is started. At the step 614, the cooldown system 300 may display the session end icon 410. For example, the communication screen 136 may display the session end icon 410 while the device 100 is powering off prior to displaying any information related to the cooldown time. The session end icon 410 may be displayed on the communication screen 136 for a duration of the first holdoff timer 326. The session end icon 410 may indicate to a consumer that the session is ending and that the cooldown timer 322 is being activated. The session end icon 410 may be displayed on the communication screen 136 prior to any information indicating a status of the cooldown timer 322.

After the first holdoff timer 326 is activated at the step 612 and the session end icon 410 is displayed at the step 614, the method 600 may proceed to conditional step 616. At the conditional step 616, the cooldown system 300 may determine whether the first holdoff timer 326 has elapsed. If the first holdoff timer 326 has not elapsed, the method 600 may return to the step 614 and the processor 302 may continue to display the session end icon 410. If the first holdoff timer 326 has elapsed, the method 600 may proceed to step 618.

At the step 618, the cooldown system 300 may be configured to calculate a percentage of time remaining on the cooldown timer 322. In some embodiments, the cooldown system 300 may additionally display an icon, such as the timer icon 402, corresponding to the percentage of time remaining on the cooldown timer 322 on the communication screen 136.

The method 600 may proceed to step 620 after the icon corresponding to the percentage of time remaining on the cooldown timer 322 is displayed on the communication screen 136. At the step 620, the refresh timer 324 may be started. The refresh timer 324 may be configured to measure a refresh time. After the refresh time elapses, the icon corresponding to the percentage of time remaining on the cooldown timer 322 may be updated to reflect a new percentage of time remaining on the cooldown timer 322. In some embodiments, the refresh time may be about six seconds such that the timer icon 402 may be updated about 20 times while the cooldown timer 322 is active.

After the refresh timer 324 is started at the step 620, the method 600 may proceed to conditional step 622. The conditional step 622 may determine whether the refresh timer 324 has elapsed. If the refresh timer 324 has elapsed, the method 600 may proceed to conditional step 624 where the cooldown system 300 determines if the cooldown timer 322 has elapsed. If the cooldown timer 322 has not elapsed, the method 600 may proceed back to the step 618 where the percentage of time remaining on the cooldown timer 322 may be recalculated and the icon corresponding to the percentage of time remaining on the cooldown timer 322 may be updated and displayed on the communication screen 136. If the cooldown timer 322 has elapsed, the method 600 may proceed to step 626 where the cooldown system 300 is configured to display the capsule eject icon 408 on the communication screen 136. The capsule eject icon 408 may indicate to a consumer that the capsule 214 may be removed from the device 100 because the device 100 has reached a comfortable or predetermined temperature proximate to the capsule 214. After the step 626, the method 600 may end with the device 100 returning to normal operation.

If the cooldown system 300 determines that the refresh timer 324 has not elapsed at the conditional step 622, the method 600 may proceed to conditional step 628 where the cooldown system 300 determines if the device 100 is being turned off. In some embodiments, the device 100 may be turned off by a consumer pressing the control button 138 to manually power off the device 100. Additionally or alternatively, the device 100 may be turned off if the battery monitoring system 312 determines that the deice 100 has entered a low battery state. If the cooldown system 300 determines that the device 100 is not being turned off, the method 600 may return to the conditional step 622 where the cooldown system 300 determines whether the refresh timer 324 has elapsed.

If the cooldown system 300 determines that the device 100 is being turned off at the conditional step 628, the method 600 may proceed to step 630 where the second holdoff timer 328 is started by the processor 302. The second holdoff timer 328 may be configured to measure the second holdoff time. The second holdoff time may be an amount of time that the communication screen 136 may display the first power off icon 412 and the second power off icon 413 or an indication after the control button 138 has been pressed to manually power off the device 100.

After the second holdoff timer 328 is started at the step 630, the method 600 may proceed to step 632 where the cooldown system 300 is configured to display at least one power off icon such as the first power off icon 412 and the second power off icon 413 on the communication screen 136. The first power off icon 412 and/or the second power off icon 413 may be a symbol or message that can be displayed to let the consumer know that the control button 138 has been pressed to turn the device 100 off. More specifically, the first power off icon 412 may be displayed while a consumer is pressing the control button 138. The second power off icon 413 may be displayed after the consumer presses the control button 138 for a second time to confirm that the device 100 should be powered off.

After the first power off icon 412 and the second power off icon 413 are displayed at the step 632, the method 600 may proceed to conditional step 634. At the conditional step 634, the cooldown system 300 may determine whether the second holdoff timer 328 has elapsed. If the second holdoff timer 328 has not elapsed, the method 600 may return to the step 632 where either the power off icon 412 or the second power off icon 413 continues to be displayed.

If the cooldown system 300 determines that the second holdoff timer 328 has elapsed, the method 600 may proceed to step 636 where the cooldown system 300 is configured to display the cooldown aware icon 414. The cooldown aware icon 414 may indicate that the cooldown system 300 is still operating after the control button 138 has been pressed. The cooldown aware icon 414 may also indicate to the consumer that the capsule 214 should not be removed from the device 100 because the device 100 may still be too hot to remove the capsule 214.

After the cooldown aware icon 414 is displayed at the step 636, the method 600 may proceed to conditional step 638 where the cooldown system 300 is configured to determine whether the cooldown timer 322 has elapsed. If the cooldown timer 322 has not elapsed, the method 600 may return to the step 636 and may continue to display the cooldown aware icon 414.

If the cooldown system 300 determines that the cooldown timer 322 has elapsed, the method 600 may proceed to step 640 where the communication screen 136 is turned off. When the communication screen 136 is turned off, no light, icons, or other indications may be displayed on the communication screen 136. Once the communication screen 136 is turned off, the method 600 may end with the device 100 being powered off.

In some embodiments, at any point during the method 600, the fault icon 416 may be displayed on the communication screen 136 if the device 100 entered into a fault state. The cooldown system 300 may continue to operate the cooldown timer 322 while the fault icon 416 is shown on the communication screen 136. The fault icon 416 may be displayed on the communication screen 136 until the fault is corrected. If the fault is corrected, the cooldown system 300 may return to normal operation of the method 600 and may display the appropriate icon of FIGS. 4A to 4G to indicate the state of the cooldown system 300 and of the method 600.

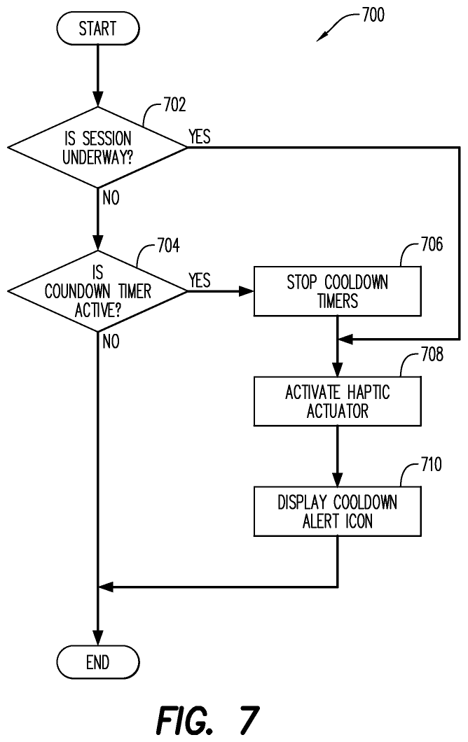

Referring to FIG. 7, a method 700 of operation of the device 100 when the lid 104 is opened is shown. The method 700 may start when the lid 104 of the device 100 is opened. When the lid 104 is opened, the method 700 may proceed to conditional step 702 to determine whether a session is ongoing. In some embodiments, determining whether a session is ongoing may be determined by determining if the processor 302 and/or the memory 304 of the cooldown system 300 has set the session underway flag 321. If the session underway flag 321 is set, a session of the device 100 may be ongoing and if the session underway flag 321 is not set, the device 100 may not have an active session underway.

If at the conditional step 702 the processor 302 determines that there is not an active session underway, the method 700 may proceed to conditional step 704 to determine whether the cooldown timer 322 of the cooldown system 300 is active. If the cooldown timer 322 is active, it may not be ideal to have the lid 104 of the device 100 in an open position because the capsule 214 may be too warm for a consumer to touch. If the cooldown timer 322 is not active, the method 700 may end with the processor 302 returning the device 100 to normal operation which may be maintaining the device 100 in the state it was in when the method 700 began.

If the cooldown timer 322 is active, the method 700 may proceed to step 706. At the step 706, the cooldown timer 322 or any other timer of the cooldown system 300 may be stopped. The cooldown timer 322 and any other timers associated with the cooldown system 300 may be stopped because the timers are used to track a time when it is optimal for a consumer to open the lid 104 of the device. If the lid 104 of the device 100 is opened, the cooldown timer 322 and any additional timers of the timer 320 are no longer necessary because the lid 104 is already opened.

After stopping the cooldown timer 322 and any additional timers at the step 706, the method 700 may proceed to step 708 where the haptic actuator 316 is actuated. Additionally, if at the conditional step 702 the processor 302 determines that a session is underway, the method 700 may proceed to the step 708. In some embodiments, the haptic actuator 316 may be configured to vibrate in a vibration pattern, such as three vibrations in a row, when the lid 104 is opened while the cooldown timer 322 is active. When the consumer feels the device 100 vibrate when the haptic actuator 316 is actuated, the consumer may be alerted that the device 100 may not be in an ideal state for the lid 104 to be opened. When the haptic actuator 316 is actuated, the consumer may be given a warning to not touch the capsule 214 since the lid 104 of the device 100 has already been opened.

After the haptic actuator 316 is actuated at the step 708, the method may proceed to step 710 where the alert icon 404 is displayed on the communication screen 136. The alert icon 404 may indicate to the consumer that the device 100 may not be at an appropriate or comfortable temperature for the consumer to touch the capsule 214 but that the cooldown system 300 has stopped the cooldown timer 322 and any additional timers of the cooldown system 300.

After the cooldown alert is displayed at the step 710, the method 700 may end by returning the device 100 to normal operation. In some embodiments returning the device 100 to normal operation may be maintaining the device 100 in the state it was in when the method 700 began.

The systems, apparatuses, and methods described herein may provide significant advantages. The cooldown system 300 may make may provide additional communication about a state of the device 100 for adult consumers. For example, the cooldown system 300 may display one or more icons to a consumer on the communication screen 136 indicating when the capsule 214 of the device 100 may be removed from the device 100. The cooldown system 300 may alert a consumer if the device 100 is too hot to be comfortably handled so that the consumer can comfortably enjoy the device 100.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

What is claimed is:

1. A cooldown system for a device, the cooldown system comprising:
at least one processor; and
a memory coupled to the at least one processor and storing instructions,
wherein the at least one processor is configured to execute the instructions to cause the cooldown system to,
detect when a session has ended,
activate a cooldown timer associated with a cooldown time,
provide an indication of a remaining time of the cooldown timer, and
in response to the cooldown time elapsing, return the device to normal operation.

2. The cooldown system of claim 1, wherein the at least one processor is configured to execute the instructions to cause the cooldown system to obtain a signal from a control button, the signal indicating that the device has been switched off and the session has ended.

3. The cooldown system of claim 1, wherein the at least one processor is configured to execute the instructions to cause the cooldown system to obtain a signal from a mechanism detection switch, the signal indicating that a lid mechanism has been opened and that the session has ended.

4. The cooldown system of claim 1, wherein the at least one processor is configured to execute the instructions to cause the cooldown system to obtain a signal from a charging connection, the signal indicating that the device is connected to a charger and that the session has ended.

5. The cooldown system of claim 1, wherein the at least one processor is configured to execute the instructions to cause the cooldown system to obtain a signal from a battery monitoring system, the signal indicating that the device has entered a low battery state and that the session has ended.

6. The cooldown system of claim 1, wherein the at least one processor is configured to execute the instructions to cause the cooldown system to detect when the device is in a fault state, the fault state indicating that the session has ended.

7. The cooldown system of claim 1, wherein the at least one processor is configured to execute the instructions to cause the cooldown system to display an alert icon on a user interface of the device if a lid mechanism of the device is opened while the cooldown timer is active.

8. The cooldown system of claim 1, wherein the at least one processor is configured to execute the instructions to cause the cooldown system to display a cooldown alert icon if a lid mechanism of the device is opened while the session is underway and prior to the cooldown timer being activated.

9. The cooldown system of claim 1, wherein the at least one processor is configured to execute the instructions to cause the cooldown system to activate a refresh timer after the cooldown timer is activated.

10. The cooldown system of claim 9, wherein the refresh timer is configured to measure a refresh time and to reset if the cooldown timer is active when the refresh time elapses, the refresh time being less than the cooldown time.

11. The cooldown system of claim 10, wherein the at least one processor is configured to execute the instructions to cause the cooldown system to refresh a timer icon when the refresh time elapses.

12. The cooldown system of claim 1, wherein the at least one processor is configured to execute the instructions to cause the cooldown system to display, on a user interface of the device, a capsule eject icon when the cooldown time has elapsed, the capsule eject icon indicating that a capsule of the device may be removed.

13. The cooldown system of claim 1, wherein the at least one processor is configured to execute the instructions to cause the cooldown system to activate a holdoff timer for a holdoff time and display, on a user interface of the device, a system icon while activating the cooldown timer.

14. The cooldown system of claim 13, wherein the at least one processor is configured to execute the instructions to cause the cooldown system to display a cooldown aware icon after the holdoff time elapses.

15. The cooldown system of claim 1, wherein the at least one processor is configured to execute the instructions to cause the cooldown system to activate a holdoff timer for a holdoff time and display a power off indication if the device is turned off while the cooldown timer is active.

16. The cooldown system of claim 15, wherein the at least one processor is configured to execute the instructions to cause the cooldown system to display a cooldown aware icon after the holdoff time elapses and until the cooldown timer has elapsed.

17. The cooldown system of claim 1, wherein the cooldown time is based on a length of a session of the device.

18. The cooldown system of claim 1, further comprising:
a temperature sensor.

19. The cooldown system of claim 18, wherein the cooldown time is based on a temperature measured by the temperature sensor.

20. The cooldown system of claim 1, wherein the cooldown timer is activated if a heater of the device or a consumable of the device is heated to a minimum threshold.

21. The cooldown system of claim 20, wherein the minimum threshold is the heater being heated for at least ten seconds.

22. The cooldown system of claim 20, wherein the minimum threshold is at least 60 Joules of energy being injected into the heater.

23. The cooldown system of claim 1, wherein the at least one processor is configured to execute the instructions to cause the cooldown system to:
set a session underway flag when the session has started; and
clear the session underway flag when the session has ended.
